(19) United States
(12) Patent Application Publication
(10) Pub. No.: US 2007/0123830 A1
(43) Pub. Date: May 31, 2007

(54) Safety syringe

(75) Inventors: Richardus Henricus Johannes Fierkens SR., Herwen (NL); Richardus Geradus Theodora Fierkens JR., Heerenberg (NL); Arnoldus Theodorus Maria Telkamp, Velp (NL); Dirk Vogelaar, Zoetermeer (NL)

Correspondence Address: WEISS & MOY PC, 4204 NORTH BROWN AVENUE, SCOTTSDALE, AZ, 85251, US

(73) Assignee: ADDINO B.V.

(21) Appl. No.: 11/289,727

(22) Filed: Nov. 29, 2005

(51) Int. Cl. A61M 5/31; A61M 5/00

(52) U.S. Cl. 604/240; 604/241

(57) ABSTRACT

A safety syringe is provided having a retractable needle assembly. The retractable needle assembly includes a hub that releasably connects to the distal end of the syringe barrel. An anchor inside the hub is fixedly connected to the needle. The anchor includes a plurality of flexible legs adapted for coupling to the plunger when the plunger is fully displaced to its distal end position within the syringe barrel. The anchor also includes a plurality of notches extending outwardly from the flexible legs. When the plunger is retracted, the plunger carries the needle and anchor assembly (the anchor and needle fixedly connected thereto) along inside the barrel as a result of the coupling between the end portion of the plunger and the anchor. The needle is therefore harbored inside the barrel for safe transport and disposal, thus minimizing needlestick injuries and reuse of the needle.

### FIELD OF THE INVENTION

This invention relates generally to medical devices and more particularly to an improved hypodermic syringe for safely administering injections.

### BACKGROUND OF THE INVENTION

Needlestick injuries have become of serious concern because of possible transmission of blood borne diseases. Blood borne diseases may also be transmitted by sharing and reusing needles. Syringes have been developed to protect a user from needlestick injuries by retracting the needle into the syringe after a hypodermic injection is performed. See, for example, U.S. Pat. Nos. 4,950,241 and 5,538,507. These syringes, however, have certain shortcomings.

Accordingly, there is a need for an improved syringe that may be used for safe and accurate injections. There is another need for an improved syringe that substantially reduces the chance of contamination through needlestick injuries. There is a still further need for an improved syringe that contains the contaminated needle after injection and renders it inaccessible for reuse. The present invention fulfills these needs and provides other related advantages.

### SUMMARY OF THE INVENTION

The present invention resides in an improved hypodermic syringe that substantially prevents needlestick injuries and reuse of the needle. The syringe comprises, generally, a cylindrical barrel releasably connected with a hub of a hypodermic needle assembly, the hypodermic needle assembly comprising an anchor fixedly attached to a hypodermic needle and releasably mounted inside the hub, and a plunger slideably mounted in the cylindrical barrel. In the process of injection, a mechanical coupling is effected between the plunger and the anchor. When the plunger is retracted proximally from the barrel after an injection, the needle is carried by the anchor inside the barrel as a result of the coupling between the anchor and the plunger.

The anchor has flexible legs capable of gripping the plunger, by means of outward facing snap elements at the proximal end of the flexible legs that engage a retaining snap edge in the plunger. The plurality of flexible legs also include stop means to substantially prevent the hypodermic needle and anchor assembly from moving into the barrel during the penetrating force of the needle into the patient.

Other features and advantages of the present invention will become apparent from the following more detailed description, taken in conjunction with the accompanying drawings which illustrate, by way of example, the principles of the invention.

### BRIEF DESCRIPTION OF THE DRAWINGS

The accompanying drawings illustrate the invention. In such drawings:

FIG. 1 is a perspective view of a hypodermic needle assembly according to the invention;

FIG. 2 is a sectional view of the hypodermic needle assembly of FIG. 1, illustrating an anchor releasably mounted in a hub and a hypodermic needle threaded through and fixedly attached to the anchor;

FIG. 2A is a sectional view of the anchor of FIG. 2, illustrating a substantially cylindrical distal portion and a plurality of flexible legs terminating in outwardly facing snap elements and including stop means;

FIG. 2B is a similar view to FIG. 2, the enlarged encircled portion illustrating a sealing lip defined between the hub and anchor;

FIG. 3 is a perspective view of a syringe including the hypodermic needle assembly of FIGS. 1 and 2 releasably mounted on the distal end of a syringe barrel with a plunger slideably mounted into the syringe barrel;

FIG. 4 is a sectional view of the syringe barrel and plunger of FIG. 3;

FIG. 5 is a partial sectional view of the syringe of FIG. 3, illustrating a distal portion of the plunger within a portion of the syringe barrel;

FIG. 6 is a similar view to FIG. 5, illustrating a more distal position of the plunger;

FIG. 7 is a similar view to FIGS. 5 and 6, illustrating the distal portion of the plunger at a fully distal position within the syringe barrel with the flexible legs of the anchor within the plunger;

FIG. 8 is a similar view to FIGS. 5-7, illustrating the outwardly facing snap elements of the flexible legs engaged with a snap edge of the plunger;

FIG. 9 is a similar view to FIGS. 5-8, illustrating full retraction of the plunger, the anchor and needle pulled inside the syringe barrel;

FIG. 10 is a perspective view of the syringe barrel of FIG. 4, with a portion of the barrel cutaway to reveal the distal portion of the plunger;

FIG. 11 is a perspective view of the plunger;

FIG. 12 is an enlarged partial perspective view of the distal portion of the plunger, illustrating the snap edge of the plunger;

FIG. 13 is another enlarged partial perspective view of the distal portion of the plunger; and

FIG. 14 is a sectional view of the syringe of FIG. 3, illustrating the plunger at different working steps within the syringe barrel.

### DETAILED DESCRIPTION OF THE PREFERRED EMBODIMENTS

As shown in the drawings for purposes of illustration, the present invention is concerned with an improved syringe, generally designated in the accompanying drawings by the reference number 10. The syringe 10 comprises, generally, a cylindrical barrel 12 having a conical distal portion 13 for releasable connection with a hypodermic needle assembly 14, the hypodermic needle assembly 14 comprising a needle 16 fixedly attached to an anchor 18 adapted for coupling to a plunger 20 slideably mounted in the barrel 12 so that when the plunger 20 is retracted proximally from the barrel 12, the needle 16 is carried by the anchor 18 (hereinafter “needle and anchor assembly”) inside the barrel 12 as a result of the coupling between the anchor 18 and the plunger 20. As used herein, the phrase “needle and anchor assembly” refers to the needle carried by the anchor.

The coupling mechanism according to the invention for effecting the coupling between the anchor 18 and the plunger 20 comprises the anchor with a plurality of flexible legs 22 terminating proximally with outward facing snap elements 24 that snap under and engage a snap edge 26 in a distal end 29 of the plunger 20.

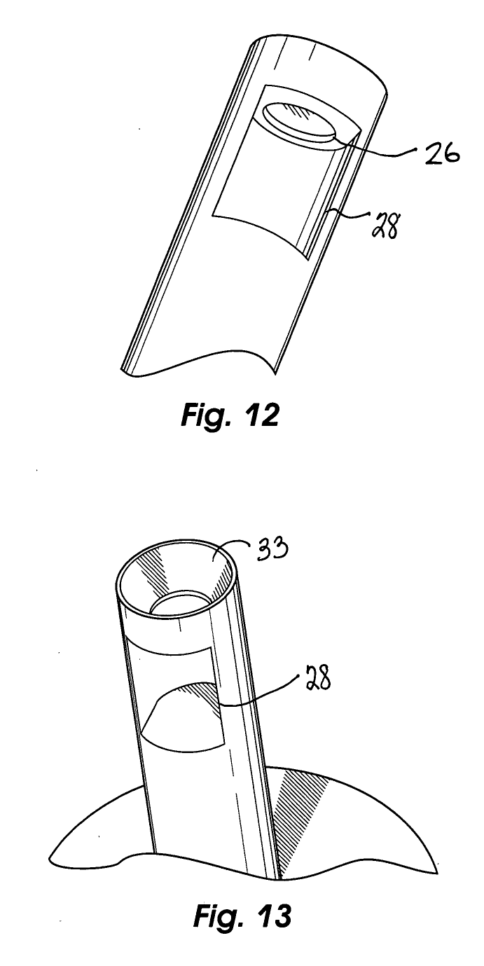

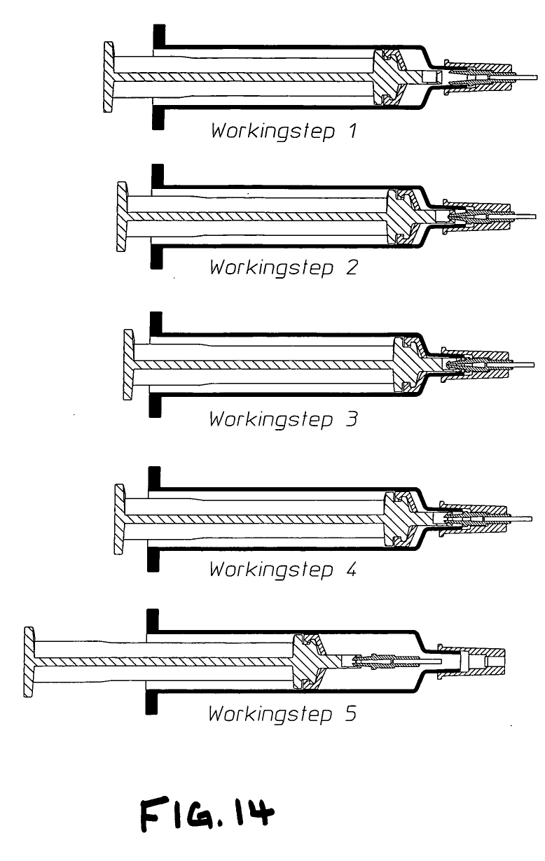

In accordance with the present invention, and as illustrated with respect to a preferred embodiment in FIGS. 1-14, the syringe 10 enables a user to safely and accurately administer injections while substantially reducing the risk of needlestick injury. The improved syringe is simply constructed, and relatively inexpensive. The improved syringe requires no change to current safety syringe injection techniques. After injection, the syringe barrel harbors the needle post-retraction for safe transport and disposal thus rendering the needle inaccessible for reuse.

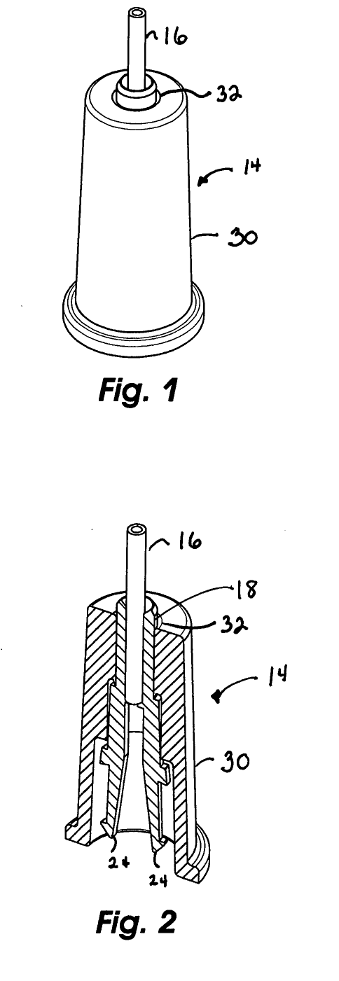

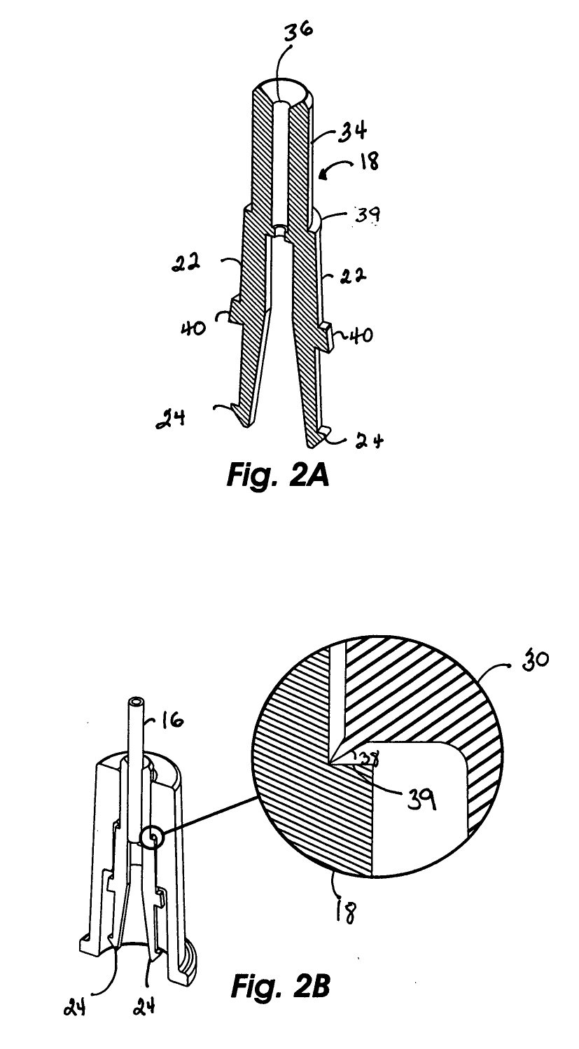

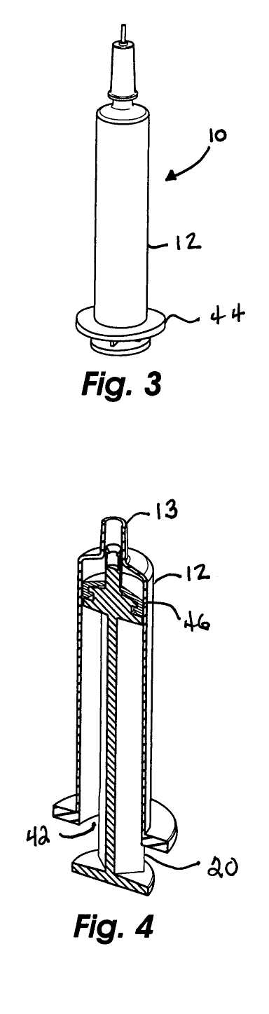

As shown in FIGS. 1-2B, the hypodermic needle assembly 14 comprises a conforming hub 30 that may be pressed against the barrel conical distal portion 13 (FIG. 3) for frictional and releasable connection therewith. The anchor 18 may be releasably mounted coaxially in the hub and the needle 16 threaded through and fixedly attached to the anchor 18. In typical manner, the needle is provided on its distal extremity with a sharpened penetrating end (not shown) in order to be able to insert the needle in the body.

As shown in FIGS. 1, 2 and 2B, the hub 30 contains a longitudinal distal bore 32 that makes it possible to close-fittingly mount it around a distal portion of the anchor. The hub defines a sealing lip 38 that substantially prevents fluid leakage between the anchor 18 and hub 30. As shown in FIG. 2A, the anchor 18 comprises a substantially cylindrical distal portion 34 containing a longitudinal bore 36 through which the needle 16 is threaded and fixedly attached and a proximal portion defined by the plurality of flexible legs 22. The diameter of the bore 36 in the anchor 18 corresponds to the diameter of the needle threaded therethrough to fix the needle in the bore without substantially any clearance between the bore and the needle. Anchor bore diameters may thus be sized to permit needles with different diameters to be placed therein.

As shown in FIGS. 2A and 2B, the anchor 18 extends radially outwardly from the substantially cylindrical portion of the anchor to define a radial edge 39 before extending proximally to the plurality of flexible legs. The plurality of flexible legs 22 terminate in the outward facing snap elements 24 and include outward facing notches 40 in approximately the middle of the flexible legs for purposes as hereinafter described. As shown in FIGS. 5-8, the outward facing notches 40 are disposed inside the hub between the distal end of the syringe barrel and the substantially cylindrical part of the anchor until the needle and anchor assembly are retracted as hereinafter described. During forward movement of the plunger as a result of the distally oriented force exerted on the needle, the radial edge 39 of the anchor becomes blocked by the sealing lip 38 to substantially prevent movement of the needle and anchor assembly from the barrel to avoid the needle from penetrating further into the body of the patient

As is known in the art, the syringe barrel 12 has the conical distal end 13 that may be formed concentric to the axis of the barrel and a proximal open end 42. A flange 44 may extend radially from the proximal open end to prevent slipping of the barrel through the finger.

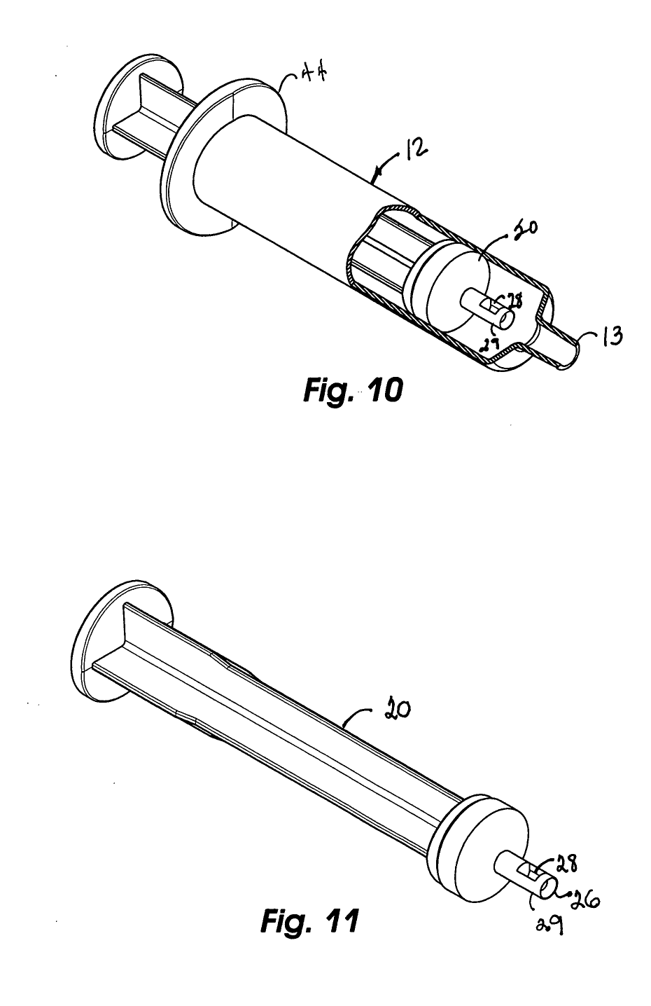

As shown in FIGS. 10-13, the plunger 20 has a substantially cylindrical distal end portion 29 having a small recessed opening 28 in a side thereof and an inward funnel 33 at the tip for guiding the plurality of flexible legs of the anchor into the plunger. A substantially circular opening 35 at the bottom of the narrower part of the funnel 33 defines the snap edge 26 for coupling with the snap elements 24 of the anchor.

The substantially cylindrical distal end portion 29 of the plunger 20 is received in the distal end portion of the barrel 13 when the plunger 20 is at its distal position as shown in FIG. 4. A plunger gasket 46 substantially prevents leakage of the syringe. A medication or the like (not shown) for injection is disposed inside the barrel 12 between the plunger gasket 46 and the distal end of the barrel 13.

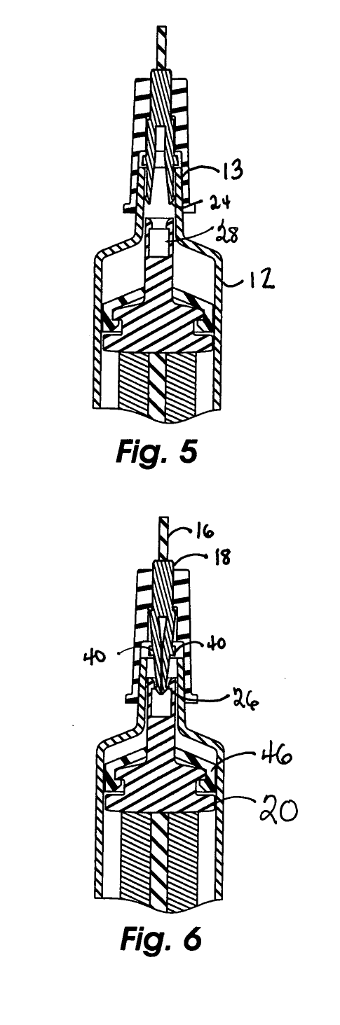

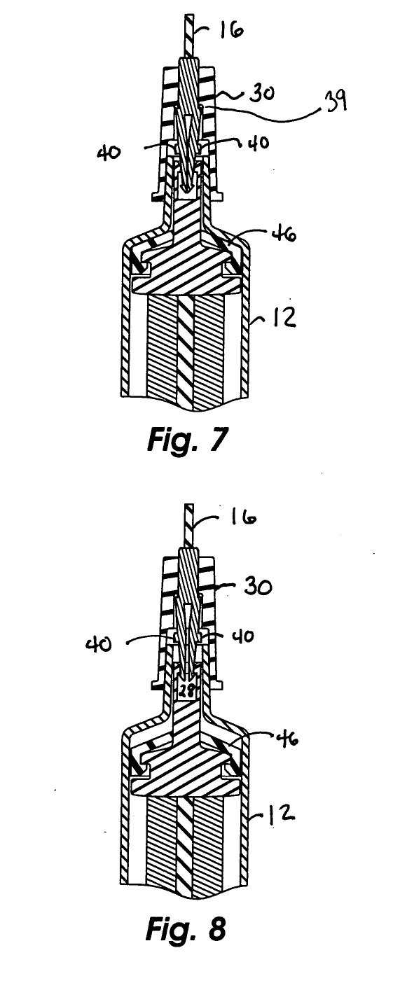

In use, a suction needle (not shown) is placed on the syringe 10 by sliding a conventional needle hub (not shown) onto the distal portion of the barrel 13. The suction needle (not shown) can be placed in the liquid and the injection liquid drawn into the syringe by retracting the plunger 20. After removing the conventional needle hub from the distal portion of the syringe barrel, the hypodermic needle assembly 14 is placed on the syringe barrel 12 by sliding the hub 30 onto the distal end of the syringe barrel 13. The medication is then administered to the patient in the usual way, by inserting the distal extremity of the needle into the patient and the medication discharged into the patient by displacing the plunger 20 toward its distal end position as shown beginning in FIG. 5. Once the medication is expelled, continued displacement of the plunger 20 at its distal end position pushes the plunger toward the snap elements 24 of the anchor 18. As the plunger is further displaced toward its distal end portion, the flexible legs 22 of the anchor bend radially inwardly because of their flexible construction as shown in FIG. 6 to fit into the distal end of the plunger 29. When the plunger is fully displaced at its distal end position as shown in FIG. 7, the snap elements 24 move past the snap edge 26 and expand to snap under the snap edge 26 of the plunger as shown in FIG. 8, thus effecting an operational coupling between the plunger 20 and the anchor 18.

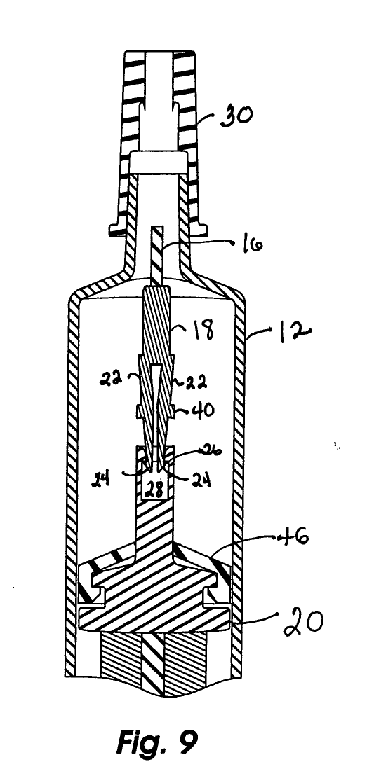

The plurality of notches 40 substantially prevent movement of the anchor and needle assembly into the barrel when there is a proximal force on the needle (i.e. the penetrating force of the needle into the patient). As shown in FIGS. 6-8, the plurality of notches are closer to each other when the anchor is coupled to the plunger thereby permitting the anchor and needle assembly to retract through the distal end portion of the barrel following penetration as shown in FIG. 9. When the plunger is retracted proximally after the liquid has been injected (See FIG. 9), the plunger carries the anchor and the needle fixedly connected (i.e. the needle and anchor assembly) thereto along inside the barrel as a result of the coupling between the end portion of the plunger and the anchor. The needle is therefore harbored inside the barrel for safe transport and disposal, thus minimizing needlestick injuries and reuse of the syringe. Each of these working steps is shown in FIG. 14.

Although a particular embodiment of the invention has been described in detail for purposes of illustration, various modifications may be made without departing from the spirit and scope of the invention. Accordingly, the invention is not to be limited, except as by the appended claims.

We claim:

1. An improved syringe comprising:
a syringe barrel having a distal end;
a hypodermic needle assembly comprising:
a hub close fittingly engaged in releasable connection with the distal end of the syringe barrel; and
an anchor releasably mounted in the hub and a hypodermic needle threaded through and fixedly attached to the anchor, the anchor including coupling means adapted for coupling to a plunger when the plunger is displaced to its distal end position; and
a plunger slideably mounted in the syringe barrel and containing at least one snap edge adapted for engagement with the coupling means.

2. The improved syringe of claim 1, wherein the coupling means comprise a plurality of flexible legs capable of gripping the at least one snap edge of the plunger.

3. The improved syringe of claim 2, wherein the plurality of flexible legs terminate at outward snap elements adapted to snap under the at least one snap edge of the plunger to effect coupling of the anchor and the plunger.

4. The improved syringe of claim 3, wherein the plurality of flexible legs each include at least one notch that extends outwardly therefrom.

5. The improved syringe of claim 2, wherein the anchor has a substantially cylindrical distal portion with the plurality of flexible legs extending proximally from a radial edge between the substantially cylindrical distal portion and the plurality of flexible legs.

6. The improved syringe of claim 5, wherein the hub defines a sealing lip between the hub and the substantially cylindrical distal portion of the anchor.

7. The improved syringe of claim 3, wherein the plunger has a substantially cylindrical distal end portion having a small recessed opening in a side thereof and an inward funnel at the tip for guiding the plurality of flexible legs of the anchor into the plunger.

8. The improved syringe of claim 7, wherein a substantially circular opening at the bottom of the narrower part of the inward funnel defines the at least one snap edge for coupling with the outward snap elements of the anchor.

9. A hypodermic needle syringe comprising:
a syringe barrel having a distal end;
a hypodermic needle assembly comprising:
a hub close fittingly engaged in releasable connection with the distal end of the syringe barrel; and
an anchor releasably mounted in the hub and a hypodermic needle threaded through and fixedly attached to the anchor, the anchor comprising a substantially cylindrical distal portion before extending proximally to a plurality of flexible legs terminating in outward facing snap elements adapted for coupling to a plunger when the plunger is displaced to its distal end position and a plurality of notches about the middle of the plurality of flexible legs; and
a plunger slidably mounted in the syringe barrel and containing at least one snap edge in a distal end portion adapted for engagement with the outward facing snap elements.

10. The hypodermic needle syringe of claim 9, wherein the anchor has a substantially cylindrical distal portion with the plurality of flexible legs extending proximally from a radial edge between the substantially cylindrical distal portion and the plurality of flexible legs.

11. The hypodermic needle syringe of claim 10, wherein the hub defines a sealing lip between the hub and the substantially cylindrical distal portion of the anchor.

12. The hypodermic needle syringe of claim 9, wherein the plunger has a substantially cylindrical distal end portion having a small recessed opening in a side thereof and an inward funnel at the tip for guiding the plurality of flexible legs of the anchor into the plunger.

13. The hypodermic needle syringe of claim 12, wherein a substantially circular opening at the bottom of the narrower part of the inward funnel defines the at least one snap edge for coupling with the outward snap elements of the anchor.
